(19) United States
(12) Patent Application Publication
(10) Pub. No.: US 2002/0111662 A1
(43) Pub. Date: Aug. 15, 2002

(54) System and method for placing an implantable medical device within a body

(76) Inventors: Paul A. Iaizzo, White Bear Lake, MN (US); Timothy G. Laske, Shoreview, MN (US)

Correspondence Address: Girma Wolde-Michael, Medtronic, Inc., MS 301, 710 Medtronic Parkway, Mailstop LC340, Minneapolis, MN, 55432, US

(21) Appl. No.: 10/072,786

(22) Filed: Feb. 8, 2002

Related U.S. Application Data
(60) Provisional application No. 60/267,687, filed on Feb. 9, 2001.

(51) Int. Cl. A61N 1/00

(52) U.S. Cl. 607/119; 607/122

(57) ABSTRACT

A system and method for positioning an implantable medical device (IMD) within a living body is disclosed. The IMD includes a flow-directed member that is deployed within the body to carry the IMD via the flow of blood. The flow-directed member may be an inflatable member such as a balloon, or a mechanical member such as a parachute structure that deploys within the body. The IMD further includes a pressure measuring device and a pressure monitor to obtain pressure measurements at one or more locations within the body adjacent the IMD. The pressure measurements are used to estimate the location of at least a portion of the IMD relative to the body to aid in positioning the IMD in the body without the use of a fluoro-visible media.

### RELATED APPLICATIONS

This application is related to, and claims the benefit of, provisionally-filed U.S. Patent Application Serial No. 60/267,687 filed Feb. 9, 2001, and entitled “Improved System and Method for Placing an Implantable Medical Device Within a Body”, which is incorporated herein by reference in its entirety.

### FIELD OF THE INVENTION

This invention relates generally to a system and method for placing implantable medical devices within a body; and more particular, relates to the use of flow-directed means to accurately place an implantable medical device within the chambers of the heart or the related vascular system.

### BACKGROUND OF THE INVENTION

Implantable medical electrical leads are well known in the fields of cardiac stimulation and monitoring, including cardiac pacing and cardioversion/defibrillation. In the field of cardiac stimulation and monitoring, endocardial leads are placed through a transvenous route to position one or more sensing and/or stimulation electrodes in a desired location within a heart chamber or interconnecting vasculature. During this type of procedure, a lead is passed through the subclavian, jugular, or cephalic vein, into the superior vena cava, and finally into a chamber of the heart or the associated vascular system. An active or passive fixation mechanism at the distal end of the endocardial lead may be deployed to maintain the distal end of the lead at a desired location.

Routing an endocardial lead along a desired path to a target implant site can be difficult. Several common approaches have been developed to accomplish this task. According to one method, a guide catheter is steered into the desired location in the vasculature. A lead is then fed through the inner lumen of the catheter such that the lead electrode(s) are positioned at the implant site. The guide catheter may then be withdrawn. This type of approach is described in commonly assigned U.S. Pat. Nos. 6,006,137, 5,246,014, and 5,851,226 incorporated herein by reference.

Locating a target location using a guide catheter can be challenging. One mechanism used to place a catheter distal tip at a desired implant site involves the use of radiopaque dye. This dye may be injected into the venous anatomy so that the chambers of the heart and the related vasculature are visible using a fluoroscopic device. This procedure, sometimes referred to as a “venogram”, allows the surgeon to locate a precise implant site when performing an implant procedure.

It may be undesirable to use fluoro visible media during an implant process for several reasons. First, some patients have adverse physical reactions when exposed to the fluoro visible dye used to obtain a venogram. Additionally, a fluoroscope of the type needed for obtaining the fluoro-visible image may not be available. This is particularly true in third-world countries where expensive medical equipment is not readily accessible. Finally, obtaining the venogram adds additional steps to the implant procedure, lengthening the time required to complete the procedure and increasing the risk of infection and complications to the patient.

What is needed, therefore, is an alternative system and method for placing implantable medical devices at precise locations within the vascular system of the body without the need to inject a fluoro visible media into the body.

### SUMMARY OF THE INVENTION

The current invention provides a system for positioning an implantable medical device (IMD) within a living body. The IMD may be a lead, a guide catheter, a sheath, or another type of device known in the art for implantation within a body. The IMD includes a flow-directed member that is deployed within the body to carry the IMD via the flow of blood. The flow-directed member may be an inflatable member such as a balloon, or a mechanical member such as a parachute structure that deploys within the body.

In one embodiment of the invention, the flow-directed member, which generally is coupled to a distal portion of the IMD, is deployed after the IMD distal portion is introduced into the right atrium of a heart. The flow of blood may be allowed to carry the distal portion through the tricuspid valve, into the ventricle, and even further through the pulmonary valve and into the pulmonary artery if desired.

The system of the current invention further includes a pressure measuring device coupled to the IMD near the distal end of the device to measure pressure near the distal end as the device moves through the body. The pressure measuring device may include a pressure transducer located at a distal end of the device. Alternatively, the pressure measuring device may include a lumen that fluidly couples a port at the device distal end to a transducer locate elsewhere on the IMD body so that pressure may be sensed by the transducer. Measurements obtained by the pressure measuring device may be used to approximate the location of the distal end. This is possible since, within the heart and vascular system, distinct pressure zones exist that can be interpreted to accurately indicate location.

In another embodiment, additional pressure sensors may be utilized to sense pressure adjacent other portions of the IMD in addition to sensing pressure at a distal end. This may be used to determine the position of the other portions of the IMD.

The system also includes a pressure monitor coupled to the pressure measuring device to utilize the pressure measurements to derive the location estimates referred to above. The pressure monitor may include a processing circuit to compare the pressure measurements with previously-acquired pressure data. The pressure data is used to correlate the measurements to an estimated location with the heart or vascular system.

According to another embodiment of the invention, a method of positioning an implantable medical device (IMD) within a living body is provided. The IMD includes a flow-directed member and a pressure measuring device. The method comprises the steps of introducing a portion of the IMD into the living body, deploying the flow-directed member, utilizing the pressure measuring device to obtain one or more pressure measurements, and utilizing the one or more pressure measurements to position the IMD within the living body.

Other aspects of the invention will become apparent from the following description and the accompanying drawings.

### BRIEF DESCRIPTION OF THE DRAWINGS

FIG. 1 is a plan view illustrating a delivery catheter having a flow-directed member located at the catheter distal tip.

FIG. 2 is a diagram illustrating placement of catheter within the pulmonary artery of a vascular system.

FIG. 3 is a diagram of a patient's heart illustrating placement of a lead via the catheter of the current invention.

FIG. 4 is a cross-sectional view of multi-lumen catheter at line 4-4 of FIG. 1.

FIG. 5 is a cross-sectional view of another embodiment of catheter.

FIG. 6 is a cross-sectional end view of catheter at line 6-6 of FIG. 1.

FIG. 7A is a side plan view of an implantable lead according to the current invention.

FIG. 7B is a view of a heart illustrating implantation of another embodiment of the implantable lead according to the current invention.

FIG. 8A is a cross-sectional end view of one embodiment of the lead at line 8-8 of FIG. 7.

FIG. 8B is a cross-sectional end view of another embodiment of the lead at line 8-8 of FIG. 7.

FIG. 10 is a plan view of a sheath adapted for use in accordance with the current invention.

FIG. 11 is a cross-sectional view of the sheath of FIG. 10 at line 11-11 of FIG. 10.

FIG. 12 is a circuit block diagram of one embodiment of the pressure monitor used according to the current invention.

FIG. 13 is a method of placing an implantable medical device within a body according to the current invention.

### DETAILED DESCRIPTION OF THE INVENTION

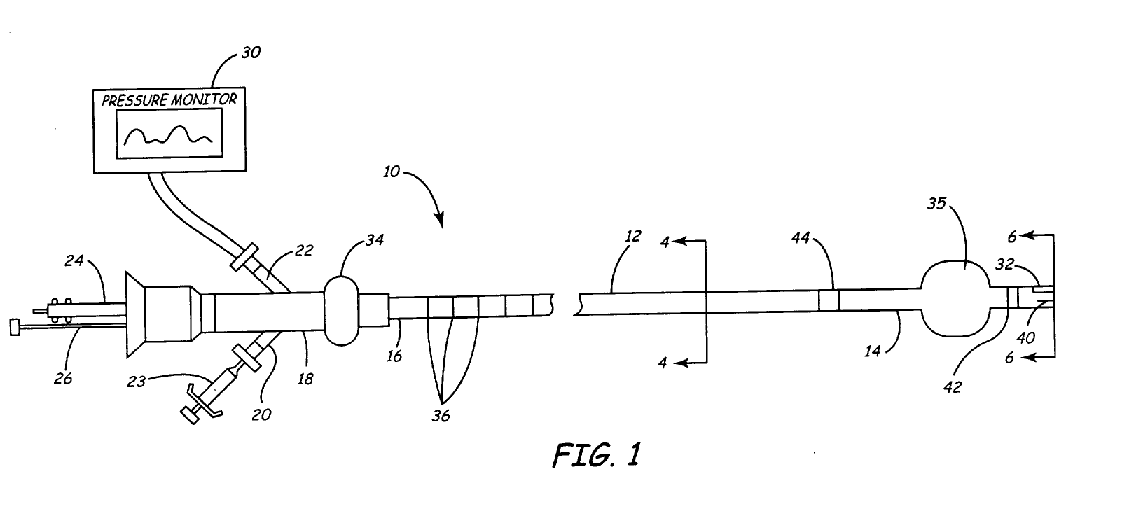

FIG. 1 is a plan view illustrating a delivery catheter 10 having a flow-directed member located at the catheter distal tip. Catheter 10 includes an elongated tubular body 12 having a distal end 14 and a proximal end 16. Tubular body 12 may be formed of silicone rubber, a polymer such as polyurethane, or any other biostable, biocompatible polymer known in the art. Distal end 14 may be formed of a material that is less stiff than proximal end 14 to provide an atraumatic distal tip section.

Tubular body 12 is coupled at the proximal end 16 to a handle structure 18. Handle structure may include one or more side arms 20 and 22. At least one of the side arms has a device such as a luer lock fitting adapted to receive a syringe 23. Handle structure further includes a lumen (not shown) which is adapted to receive a lead 24. The lead may be advanced within a lumen of tubular body in a manner to be discussed below.

Handle may further include a port for receiving a stiffening member such as stylet 26, which may also be advanced within another lumen of the tubular body, as will be described in the following paragraphs. One example of a stylet which may be used for delivery of catheter 10 is disclosed in commonly-assigned U.S. Pat. No. 4,350,169 to Dutcher et al., incorporated herein by reference. Alternatively, a stylet having a pull-wire to deflect the distal tip may be utilized, such as that disclosed in U.S. Pat. No. 5,873,842 to Brennan et al., incorporated herein by reference. In another embodiment, a stylet with a shapeable tip may be used to steer distal end 14 to the desired location of implant. For example, commonly assigned U.S. Pat. No. 4,381,013 to Dutcher is directed to the use of a two-piece stylet that enables a shape to be imparted to the lead to facilitate introduction into a predetermined implant site. The stylet includes a tubular portion that enables torque applied at the proximal end to be transmitted to a fixation means located on the distal end of the lead. Use of a stylet in placing distal end 14 of catheter 10 will be discussed further below.

Catheter 10 may include one or more pull wires that are coupled to the distal tip. Applying tension to the pull wires causes deflection of the distal tip to allow the catheter to be navigated through the vascular system of a body. Examples of such deflection mechanisms can be found in U.S. Pat. No. 4,815,478 issued to Buchbinder et al., and U.S. Pat. No. 4,940,062 issued to Hampton et al. Another example of a pull wire system is set forth in commonly-assigned U.S. Pat. No. 6,146,338 to Gardeski et al., which is incorporated herein by reference in its entirety. As described in the '388 patent, control of the deflection wire may be provided by a spinner or knob 34. Rotation and/or longitudinal deflection of this knob controls the degree and/or direction of deflection.

According to the current invention, catheter 10 may include a pressure transducer 32 located at the distal end 14 of the delivery catheter 10. Pressure transducer 32 may be any type of pressure transducer for measuring pressure within a body, including the exemplary embodiments illustrated and described in commonly-assigned U.S. Pat. Nos. 4,485,813, 4,407,296, 6,221,024, and 4,432,372, all incorporated herein by reference in their entirety. If the foregoing embodiments, pressure transducer 32 is coupled to a pressure sensing monitor at proximal end 16 of catheter 10 via multiple conductors carried by catheter body 12. The conductors transmit electrical signals that are indicative of pressure changes sensed by the transducer. These signals are used by pressure monitor 30 to provide information to a user in a manner to be discussed below.

Alternative embodiments of the invention may include a pressure transducer that is located within catheter body 12, or at proximal end 16 of the catheter 10 rather than at distal end 14 of catheter 10. In one instance, catheter 10 may include one or more hollow lumens extending longitudinally to one or more openings near the distal end of the catheter. The lumens may be charged with a liquid such as saline solution. Changes in pressure at the distal end of the catheter are transmitted via the liquid in the lumen to a pressure transducer located at a proximal end of the catheter. The pressure measurements may then be provided to pressure monitor 30. In this embodiment, side arm 22, which is in fluid communication with the pressure sensing lumen, will be coupled to means for providing a constant flow of fluid in a manner known in the art, as well as providing signals to pressure monitor 30. This type of system is available as Model No 42684-05 from Abbott Laboratories, Chicago, Ill.

In yet another embodiment, transducer 32 is omitted, and instead a membrane is located at a distal end of a catheter. The membrane is in fluid communication with a gas-filled chamber and lumen within the catheter. The membrane is positioned such that pressure exerted against the outer surface of the membrane will cause the membrane to compress, increasing the pressure of the gas within the gas-filled chamber and associated catheter lumen. The catheter lumen is connectable to, or may incorporate, a pressure sensor to sense the changes in gas pressure within the catheter lumen. In the manner discussed above, the pressure sensor emits electrical signals in response to pressure changes that may be used by pressure monitor to provide information to a user. A system of this nature is described in U.S. Pat. No. 5,573,007 to Bobo, incorporated herein by reference.

Regardless of the mechanism used to obtain pressure measurements, electrical signals indicative of these measurements are provided to pressure monitor 30. In turn, pressure monitor provides a pressure waveform or some other type of pressure indication to the user. Pressure monitor 30 may include a user display, means for generating an audible signal, or any other type of means to communicate the data. Pressure monitor 30 is described in detail below.

Delivery catheter 10 of the current invention further includes a flow-directed member. In FIG. 1, this flow-directed member is an inflatable device such as balloon 36, which may have an inflated diameter of less than about 15 mm. The balloon is in fluid communication with side arm 20 via a lumen provided by tubular body 12. The balloon may be inflated by injecting an inflation fluid through side arm 20. The inflation fluid may be a liquid such as saline, or a gaseous mixture such as air.

The balloon may be formed of compliant or non-compliant polymer materials. Example of materials that are suitable for balloon construction include polyethelene, nylon, PET, and laytex. In one embodiment, the balloon is formed of polyurethane such as Pellethane™ having a stiffness of approximately 80A Shore which is available from World Medical of Miami, Fla. The balloon may be attached to the lead body using a medical grade adhesive, as is known in the art.

Balloon 36 is of the type known for use with a Swan-Ganz catheter or wedge pressure catheter. This balloon is inflated after the distal end 14 of catheter 10 is positioned within the right atrium of the heart. The flow of blood carries the balloon into the right ventricle and further into the pulmonary artery. By monitoring pressure indications and/or other data signals provided by pressure monitor 30, an exact location of the distal tip 40 of the catheter 10 may be determined. This information may be used to position the distal end 14 within a few millimeters of a desired location in a chamber of the heart or within the pulmonary artery. This is discussed further below.

Catheter position may be further identified using marker bands 36 provided on proximal end 16 of the catheter 10. These marker bands indicate the portion of the catheter that has been advanced within the vascular system of the patient. These marker bands may be indentations or visible markings provided on the outer surface of the catheter 10.

Catheter 10 may further include one or more electrodes such as ring electrodes 42 and 44 for sensing electrical signals and/or delivering electrical stimulus. These electrodes could be any of the various types of pacing and/or sensing electrodes known in the art. For example, one or more of these electrodes may be a porous platinized electrode assembly. In one embodiment, these electrodes may be steroid-eluting. Suitable electrode assemblies are described in commonly-assigned U.S. Pat. No. 4,506,680 to Stokes, and related U.S. Pat. Nos. 4,577,642, 4,606,118, and 4,711,251.

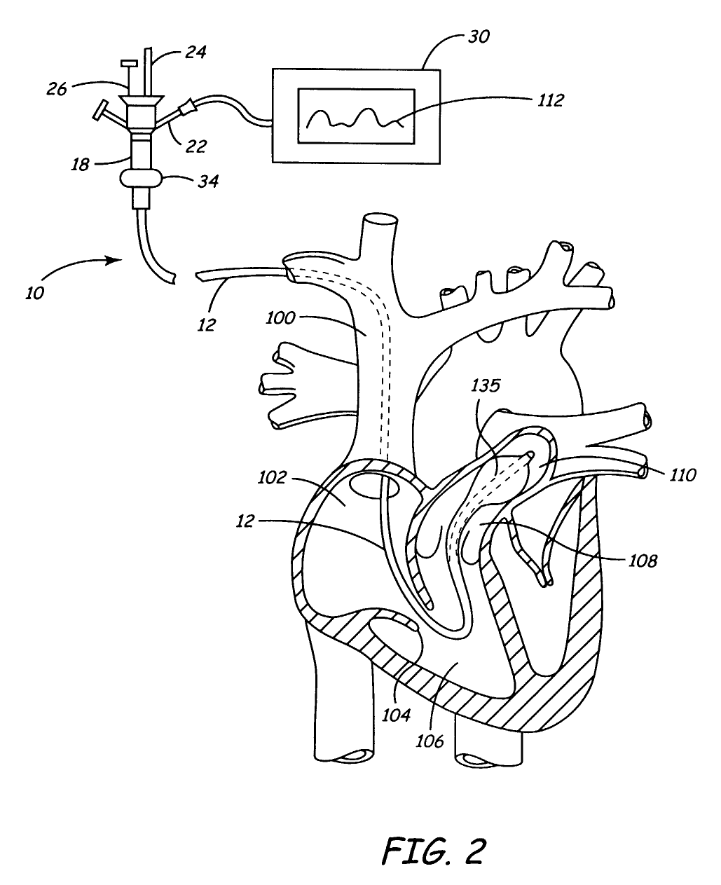

FIG. 2 is a diagram illustrating placement of catheter 10 within the pulmonary artery of a vascular system. Tubular body 12 of catheter may be introduced through a peripheral vein of a body into the superior vena cave 100 using commonly-known introduction techniques. Distal end 14 of the catheter is advanced into the right atrium 102, where the flow-directed member such as balloon 35 is inflated. If desired, the flow of blood may be allowed to carry the balloon 35 through the right atrium 102, through the tricuspid valve 104, into the right ventricle 106. The balloon may further be advanced through the pulmonary valve 108 and into the pulmonary artery 110.

Periodically throughout the procedure, pressure measurements may be obtained by transducer 32 or another pressure measuring device. The measured pressure signals may be used by pressure monitor 30 to estimate a location of a portion of the catheter within the patient's body. This is possible because intravascular pressure varies by location within the heart as well as within the associated vascular system. For example, a distinct pressure shift may be detected as a sensor is moved from one cardiac chamber to the next, and even as the sensor changes position within the cardiac chamber. Therefore, a pressure sensor coupled to an implantable medical device (IMD) may be used to measure pressure signals that may then be interpreted to estimate sensor location, and, in turn, to estimate the location of a portion of an IMD.

In the instant embodiment, the location of the distal tip of the catheter is determined using the pressure measurement. According to one manner of use, a displayed pressure signal 112 may be viewed by a user having a knowledge of typical pressure shifts that generally occur during a particular procedure. Using this knowledge, the distal tip 40 of catheter 10 may be positioned at a desired position in the right atrium or ventricle, or even within the pulmonary artery 110. For example, the catheter may be allowed to advance to the pulmonary value 108 as indicated by a predetermined pressure signal. The catheter may then be withdrawn a specific distance so that the catheter is precisely positioned within the right ventricle 106. The marker bands 36 on the proximal end 16 of the catheter may aid in precisely advancing or withdrawing the tubular body 12 a known amount.

In one embodiment of the invention, pressure monitor includes processing means to receive the pressure measurements and automatically compare the pressure measurements against stored pressure profiles. Based on the results of the comparison, a visual representation of the catheter within the patient's vascular system may be provided to the user to aid in the positioning of the catheter distal tip.

Once the catheter has been precisely located, the distal end of the catheter may be deflected using a deflectable stylet 26, or by using internal pull wires included within the catheter body. According to one embodiment, the pull wires may be manipulated via knob 34, which operates as described in the '338 patent referenced above. Upon deflection of the catheter tip, a lead 24 may be advanced within a delivery lumen and attached to the myocardium via a fixation member such as a helix provided on the lead body.

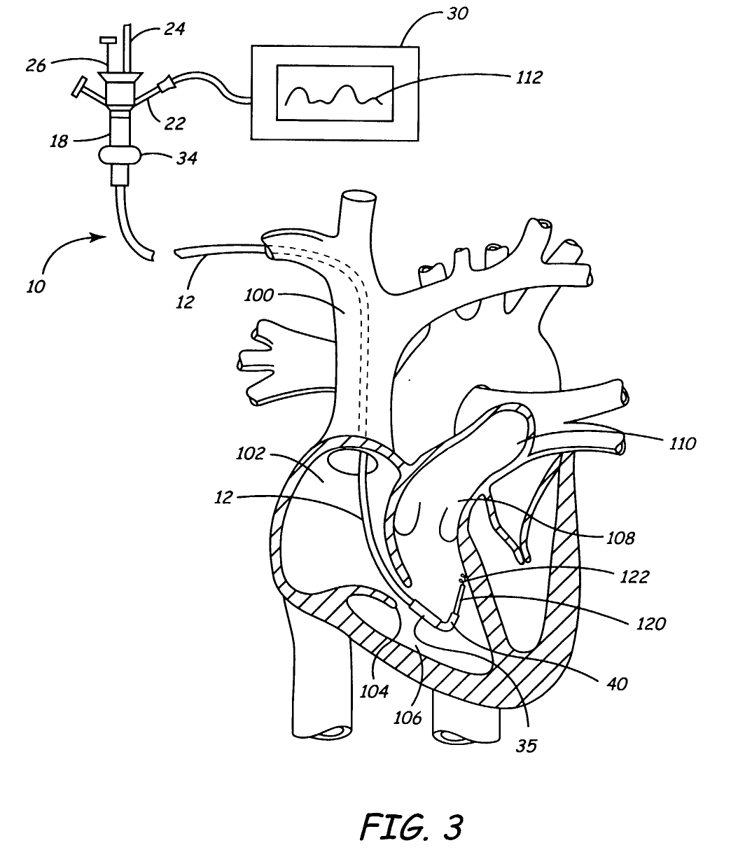

FIG. 3 is a diagram of a patient's heart illustrating placement of a lead via the catheter of the current invention. In this figure, distal tip 40 of catheter 10 has been positioned within a predetermined location within the right ventricle 106. After this positioning has occurred in the manner discussed above, balloon 35 may be deflated. A predetermined distal portion 120 of lead 24 may be advanced past the distal tip 40 of the catheter. Before, or after, the lead tip is advanced in this manner, the distal tip of the catheter may be deflected in a predetermined direction using a pull wire mechanism, or a deflectable stylet 26 as is discussed above. Once a desired deflection of the catheter tip has been achieved, a fixation member carried on the distal portion 120 of lead 24 may be utilized to attach the lead to the myocardium. In FIG. 3, lead 24 is shown having a fixation helix 122.

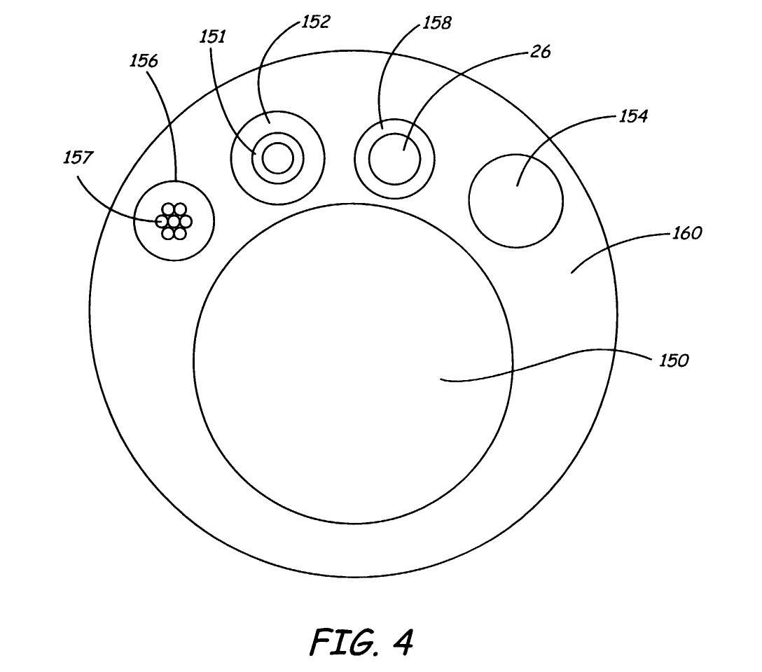

FIG. 4 is a cross-sectional view of multi-lumen catheter 10 at line 4-4 of FIG. 1. As discussed above, in this embodiment, catheter 10 includes a lead-delivery lumen 150, which opens to a port in the distal end of catheter 10. Lead 24 may be delivered through lumen 150 after the catheter has been accurately positioned within the vascular system. It may be further noted that lumen 150 may be used to receive a stiffening member when a lead is not positioned therein.

In one embodiment, catheter 10 further includes a pressure sensing lumen 152. This lumen extends from the distal tip 40 of catheter 10 to side arm 22. A pressure transducer 32 may be located at distal end 14 of catheter 10 as shown in FIG. 1. In this case, lumen 152 carries multiple conductors, which are shown as a multi-conductor coil in FIG. 4. The conductors carry electrical signals generated by pressure transducer 32, and which are provided to pressure monitor 30 in the manner discussed above. The number of conductors 151 will depend on the type of pressure sensor selected for use in the system.

As discussed above, any type of pressure sensor adapted for obtaining pressure readings within a body may be utilized as pressure transducer 32. If a pressure sensor such as described in U.S. Pat. No. 6,221,024 to Miesel is selected for use, three conductors will be carried by lumen 152. A two-conductor arrangement is described in commonly-assigned U.S. Pat. No. 4,432,372 to Munroe. Other four-conductor systems are available, such as the transducer described in U.S. Pat. No. 4,023,562 to Hynecek et al. In any case, the conductors may take the form of a multi-conductor coil as represented by FIG. 4. Alternative, the conductors may be provided as a twisted cable, as multiple, insulated concentrically-arranged coils, or in any other arrangement known in the art.

In another embodiment, lumen 152 extends from the distal tip 40 of catheter 10 to side arm 22 and may be filled with liquid or gas in the manner discussed above. In this instance, the conductors 151 are omitted. The gas or liquid within the lumen transfers pressure changes sensed via a membrane or an open lumen port, respectively, at the catheter distal end to a transducer at a proximal end of catheter. The resulting electrical signals generated by the transducer may then be provided to pressure monitor 30.

Catheter 10 also provides an inflation lumen 154 that is in fluid communication with side arm 20, and which extends to flow-directed member such as balloon 35. This lumen carries fluid, which may be liquid or gas, from a syringe inserted in side arm 20 to balloon 35. This lumen is used to inflate or deflate the balloon by injecting or withdrawing the fluid, respectively, after catheter positioning is completed.

In one embodiment, catheter 10 may include one or more lumens to carry conductors associated with electrodes. FIG. 4 illustrates lumen 156 carrying a multi-filar conductor 157 which may be coupled to one or more of the electrodes 42 and 44. The conductors may be provided in the form of either stranded or cabled conductors, as described in the '873 patent. A stranded design adaptable for use with the current invention corresponds to that disclosed in U.S. Pat. No. 5,246,014 issued to Williams et al, also incorporated herein by reference in its entirety. Other conductor types may of course also be employed, including twenty-strand cables, as described in U.S. Pat. No. 5,845,396 issued to Altman et al, also incorporated herein by reference in its entirety. In still other embodiments, a single filar wire conductor may be coiled around a second insulated conductive core member, and the two conductors may be coupled to respective electrodes to provide a bipolar application. Additional lumens for providing additional conductors may be included within catheter 10, if desired.

FIG. 4 further illustrates a lumen 158 that is optionally provided to receive a stiffening member such as stylet 26.

The various lumens shown in FIG. 4 are surrounded by a biocompatible insulative polymer 160 such as polyurethane, silicone rubber, or the like. Catheter 10 may further include a protective jacket formed of urethane, silicone, or and other biocompatible material. This jacket offers an abrasion-proof layer that increases lead stiffness to afford better pushability and torque control.

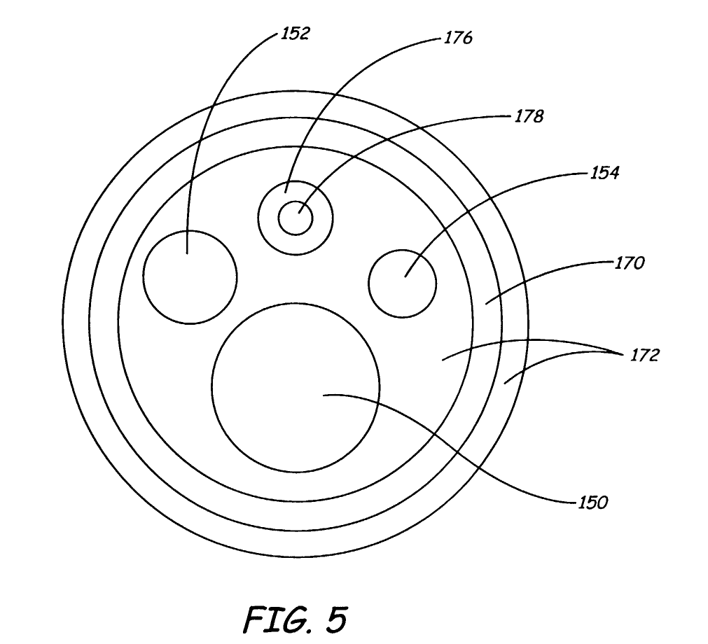

FIG. 5 is a cross-sectional view of another embodiment of catheter 10. In this embodiment, a coiled conductor 170 is embedded within the insulated polymer tubular member 172. Conductor 170 may be coupled to one or more of the electrodes 42 and 44 of FIG. 1. This embodiment further includes delivery lumen 150, pressure-sensing lumen 152, and inflation lumen 154.

The embodiment of FIG. 5 further provides a lumen 176 that carries a pull-wire 178 coupled to distal tip 40 of catheter 10. This pull-wire may take any of the forms discussed above. A preferred bending direction may be provided by selection of the location of lumen 158 as compared to the longitudinal axis of the catheter body, by positioning of other lumens within the catheter body, and/or by use of a weakened zone within the catheter body. As described with regards to FIG. 1, pull-wire deflection is controlled by a control mechanism in handle 18, such as knob 34. Multiple lumens such as lumen 176 may be provided, each to carry a respective pull-wire. In another embodiment, the pull-wires may be embedded directly within polymer tubular member. In yet another embodiment of a simplified catheter, neither pull-wires nor a stylet lumen are provided, with catheter positioning being accomplished through the ability to push and torque the catheter body itself.

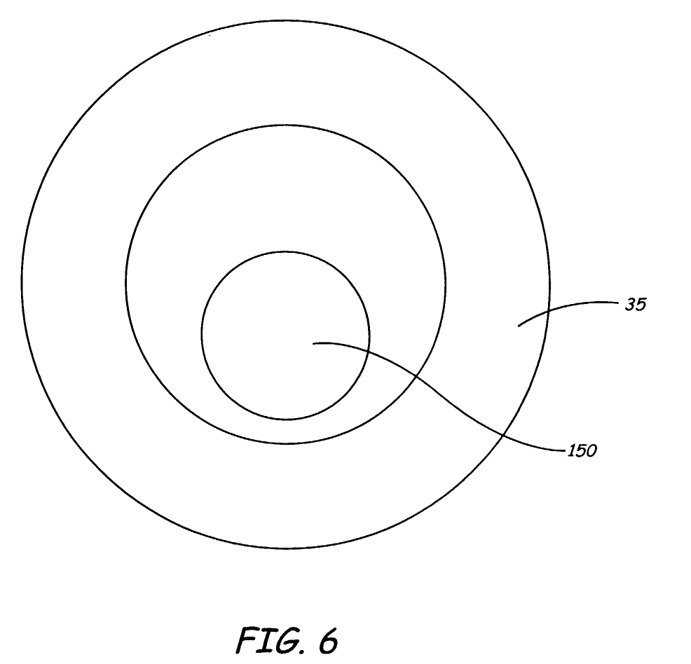

FIG. 6 is a cross-sectional end view of one embodiment of catheter 10 at line 6-6 of FIG. 1. This view shows lead-delivery lumen 150 exiting the distal tip 40 of the catheter. This view further illustrates balloon 35 in an expanded state. In another embodiment, pressure-sensing lumen 152 extends to a port at distal end 14 to facilitate pressure measurements via a column a liquid within the lumen, as discussed above.

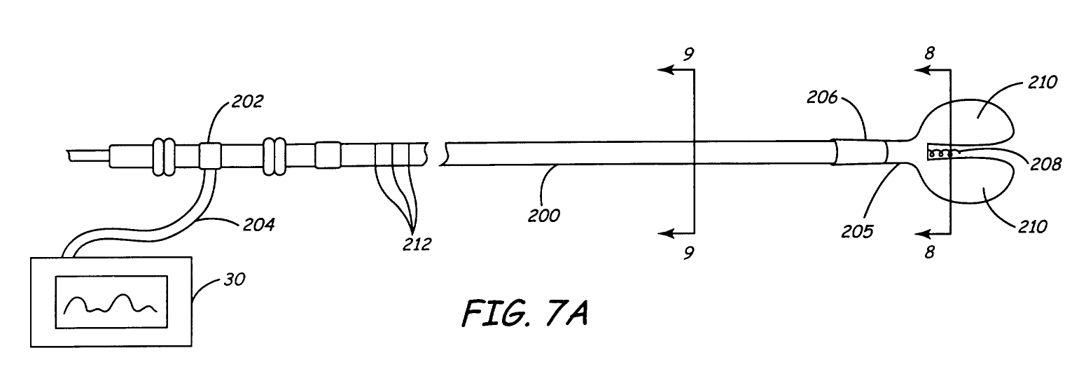

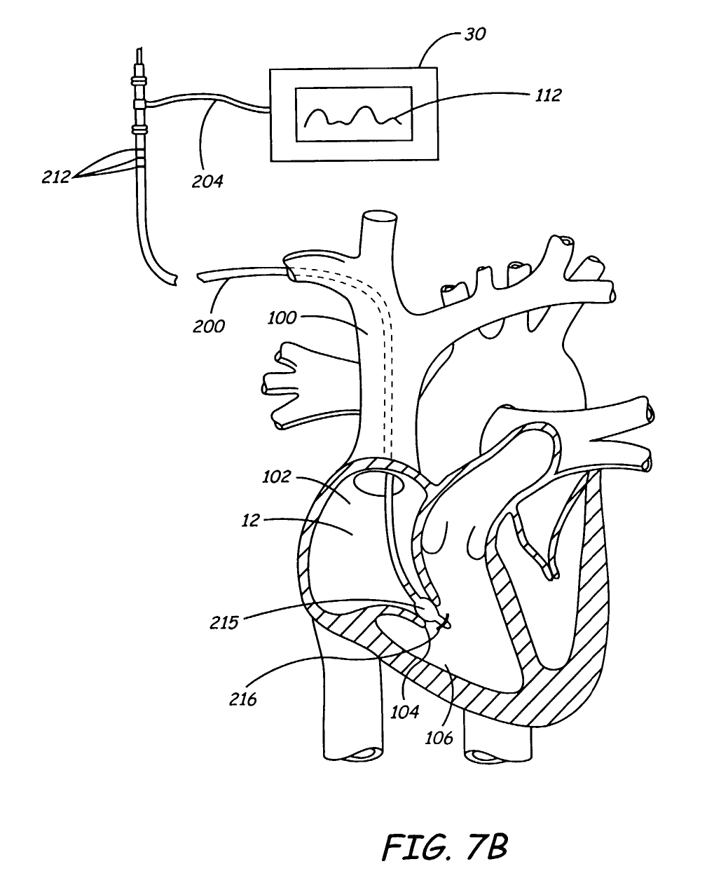

FIG. 7A is a side plan view of an implantable lead that incorporates various aspects of the current invention. Lead includes elongated insulated lead body 200 with a lead connector at the proximal end, which may take the form of any standard or non-standard connector for connecting to an implantable medical device. Lead is provided with a connector ring 202 that may be coupled via a cable 204 to pressure monitor 30. Electrical signals generated by pressure transducer 206 at the lead distal end are transferred via connector ring 202 and cable 204 to pressure monitor to be provided in format that can be understood by users. Proximal end of lead may also include marker bands 212 similar to those discussed above in reference to catheter 10 to aid in positioning the lead body.

Distal end 205 of the lead may include one or more electrodes and/or a fixation mechanism. For example, helix 208 may be employed for sensing electrical activity and/or for delivering electrical stimulation. Additionally, helix is adapted to attach to tissue within the heart as is known in the art.

According to the invention, distal end 205 of the lead includes a flow-directed member such as an inflation member 210. This member is similar to that discussed above with respect to FIG. 1, and may take any of the forms described above. In one embodiment, inflation member may be formed of a single balloon-type structure. In another embodiment, two or more balloon-like structures may be provided to surround helix 208 in the manner shown. Each of the inflation members may be in fluid communication with the same inflation lumen, or alternatively, multiple inflation lumens may be provided.

During use, the lead is introduced into the right atrium of the heart, as may be accomplished using an introducer. The inflation member 210 is inflated around the fixation helix. The inflation member is then allowed to be carried with the flow of blood to a precise location within the right atrium or ventricle by using pressure signals provided by transducer 206 and interpreted by pressure monitor 30 and, if desired, the marker bands 212 in the manner discussed above. When at the precise location, inflation member 210 may be deflated and the fixation helix attached to the myocardium. In one embodiment, lead body 200 includes a lumen to receive a stiffening member such as a stylet. The stylet may be deflectable to allow distal end 204 of the lead to be shaped in a predetermined manner prior to fixation of the helix to the heart tissue.

FIG. 7B is a view of a heart illustrating implantation of another embodiment of the lead according to the current invention. This lead includes aspects of the invention similar to those shown in FIG. 7A. Additionally, the lead includes an inflatable member 215 positioned proximal to fixation tines 216 located at the distal lead tip. The tines prove a passive means of fixation for attaching the lead distal tip to heart tissue as is known in the art.

A common problem with passive fixation mechanisms employing tines is that the tines entangle with the tricuspid valve 104 as the lead is advanced from the right atrium 102 to the right ventricle 106. This can damage the valve. The inflatable member 215 of the current embodiment prevents this from occurring by spreading the valve to allow easy passable of the lead into the ventricle. During use, the inflatable member 215 is inflated when the distal lead tip is positioned within the atrium. The flow of blood carries the lead distal tip to the tricuspid valve, where the enlarged diameter of the inflatable member dilates the valve to allow for passage of the tines. Because the tines do not extend beyond the balloon diameter, no tissue contact is made, and the lead distal tip can be carried easily into the ventricle. Thereafter, the inflatable member 215 can be deflated in the manner discussed above, and the tines can be engaged with the heart tissue.

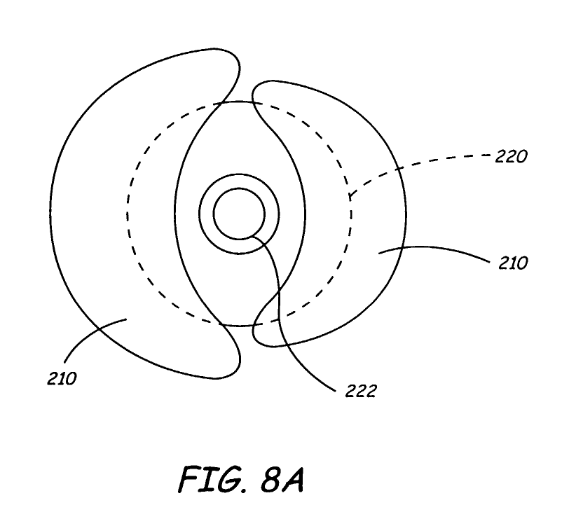

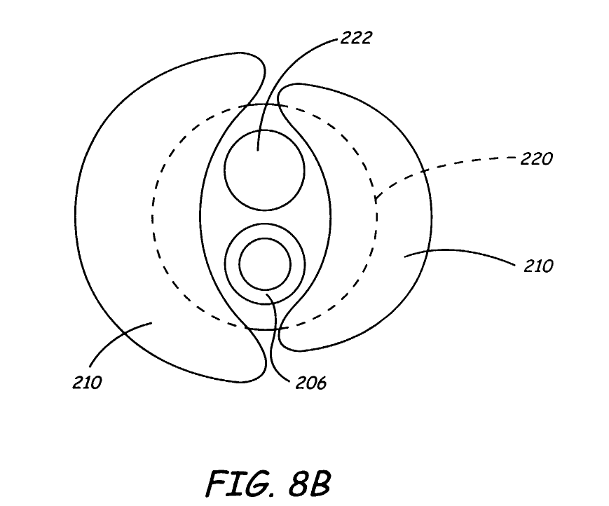

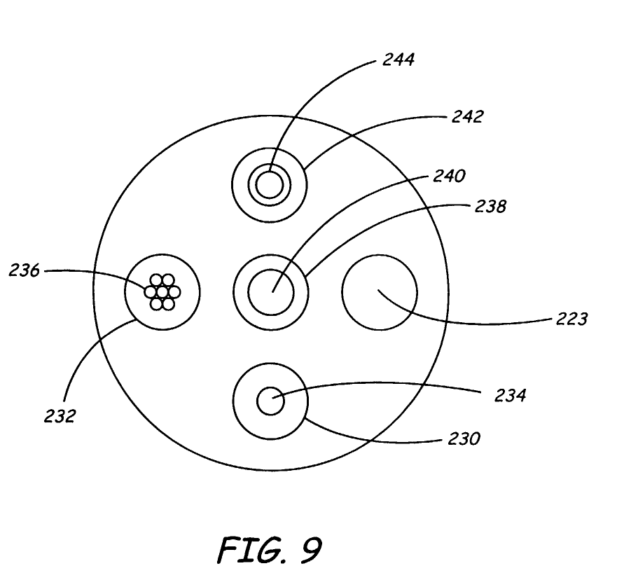

FIG. 8A is a cross-sectional end view of one embodiment of the lead at line 8-8 of FIG. 7. This view illustrates the end profile 220 (shown dashed) of the lead. This view further shows the manner in which inflation member 210 surrounds fixation helix 206 to prevent the helix from inadvertently damaging heart tissue when the lead is being positioned at the desired implant site.

FIG. 8B is a cross-sectional end view of another embodiment of the lead at line 8-8 of FIG. 7. In this embodiment, transducer 206 is located at a proximal end of lead, and is in fluid communication with bodily fluids via a pressure-sensing lumen 222. As illustrated in FIG. 8B, pressure-sensing lumen 222 opens to the body via a distal port located at the lead distal tip. Alternatively, the distal port may be provided through a side wall at a distal end of the lead. A column of saline or other fluid injected into pressure sensing lumen 222 allows the transducer to sense pressure changes in the body in the manner discussed above.

FIG. 9 is a cross-sectional end view of one embodiment of the lead at line 9-9 of FIG. 7. This embodiment, which corresponds to FIG. 8A discussed above, includes lumen 242 to carry multiple conductors 244 coupled to transducer 206. In FIG. 9, these conductors 244 are represented as a multiconductor coil, although other embodiments may be used in the manner discussed above. In a second embodiment corresponding to FIG. 8B, this lumen may instead carry fluid for use in sensing pressure changes.

The lead of FIG. 9 further includes an inflation lumen 223 coupled to control inflation of inflation member 210, and lumen 230 provided to carry a conductor coupled to helix 208. One or more additional lumens for carrying conductors may be provided to couple to additional electrodes, if desired. The lead may further include a lumen 238 for stiffening member 240, which may be a steerable stylet. This type of multiconductor, multi-lumen lead design may be of the type described in U.S. Pat. No. 5,584,873 issued to Shoberg, et al. incorporated herein by reference.

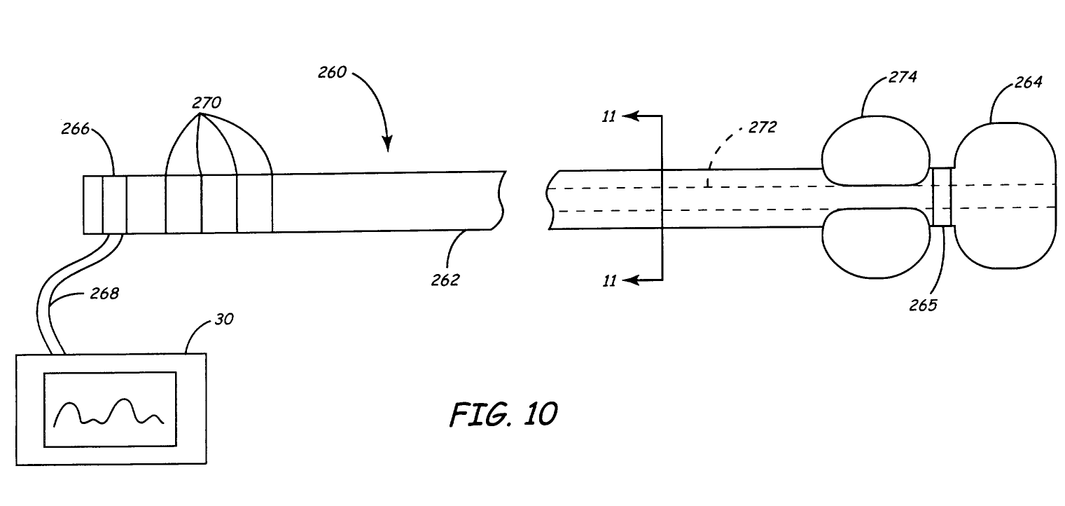

FIG. 10 is a plan view of a sheath 260 adapted for use in accordance with the current invention. The sheath body 262 may be formed of a material having sufficient stiffness to allow torque to be transferred down the sheath body so that a lead may be attached to myocardial tissue in a manner to be discussed below. Sheath 260 includes a flow-directed member 264 at the distal end, which may be an inflation member. Sheath further includes a connector such as connector ring 266 at the proximal end shown coupled to cable 268. Cable transfers one or more electrical signals generated by pressure transducer 265 to pressure monitor 30 in the manner discussed above. In response, pressure monitor 30 provides pressure indications that allow for precise positioning of the distal tip of sheath 260. Sheath may further include marker bands 270 to aid in this positioning step.

During use, sheath is positioned within the right atrium of the heart and flow-directed member 264 is inflated to allow the distal tip to be carried to a desired location. Flow-directed member may then be deflated. Before, or after, this positioning step, an implantable device such as a lead is inserted within an internal lumen 272 (shown dashed) of sheath 260. This device may then be attached to myocardial tissue in the manner discussed above.

According to one embodiment of the invention, a second inflation member 274 is provided on the sheath, which is inflated after the implantable device such as a lead is positioned within internal lumen 272. This second inflation member is adapted to compress internal lumen 272 so that the implantable device is “gripped” by the inflation member. The proximal end of the sheath 260 may then be rotated. Because of the ability to transfer torque down the sheath body, a fixation helix on the distal end of a lead positioned within lumen 272 may be readily attached to myocardial tissue. If desired, sheath may further include one or more pull-wires in the sheath walls to deflect the sheath distal tip and aid in positioning the implantable device that is positioned within lumen 272.

After an implantable device is attached to myocardial tissue, the inflation member 274 may then be deflated and the sheath removed from the body.

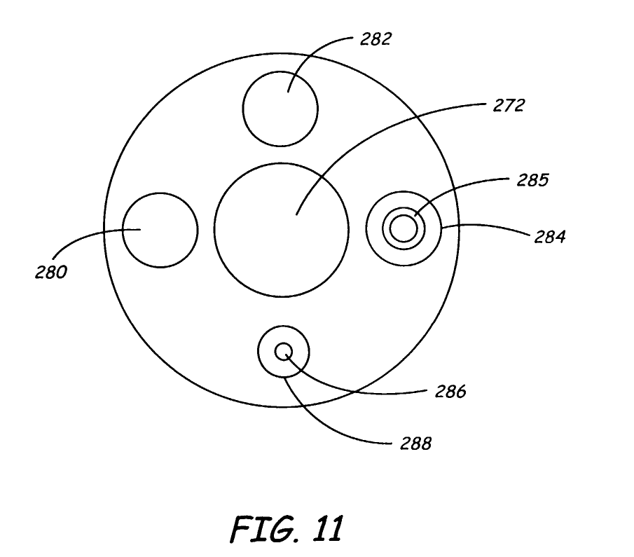

FIG. 11 is a cross-sectional view of the sheath of FIG. 10 at line 11-11 of FIG. 10. This view shows first and second inflation lumens 280 and 282, each of which is coupled to a respective one of the inflation members 264 and 274. Pressure-sensing lumen 284 is also provided to carry the multiple conductors 285 coupled to transducer 265. A pull-wire 286 within a fifth lumen 288 may also be provided in one embodiment. Additional pull-wires may be added.

The above-discussed embodiments include a flow-directed member that is an inflatable, balloon-type structure. However, it will be understood that alternative structures may be utilized. For example, catheter 10 may have a mechanical expandable member, such as a parachute or umbrella-type mechanism that is automatically expanded by its resistance to the flow of blood.

In another embodiment of the invention, catheter 10 may also include means for allowing calculation of cardiac output to be performed. For example, catheter 10 may include a first thermocouple at distal end 14, and a second thermocouple located proximal to the first thermocouples. Both thermocouples are coupled to electrical connectors at the proximal end of the catheter for measuring temperature in the pulmonary artery, thereby allowing cardiac output to be calculated using thermodilution techniques such as described in U.S. Pat. No. 4,721,115.

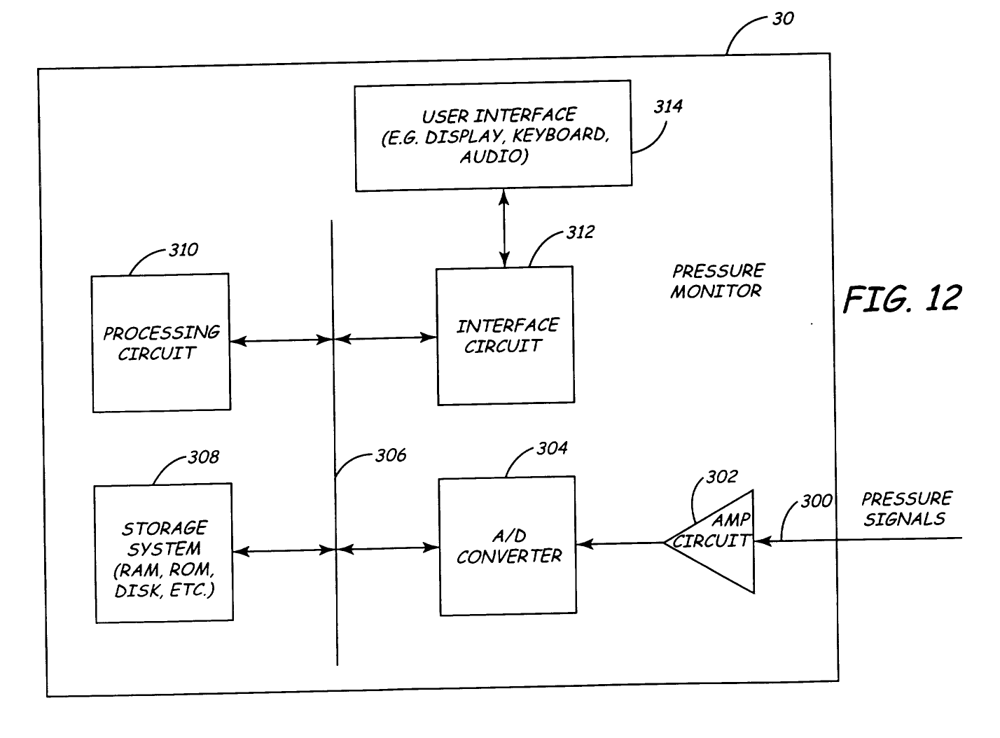

FIG. 12 is a circuit block diagram of one exemplary embodiment of pressure monitor 30, although many other embodiments of pressure monitor may be contemplated. As discussed above, a pressure transducer such as transducer 32 (FIG. 1) provides electrical signals shown on line 300. These electrical signals are indicative of the pressure measured at the distal tip of an implantable device using any of the pressure measuring configurations discussed above. Pressure signals are received by an amplifier circuit 302, which may include a filter to reduce noise signals. The amplified signals may be provided to an analog-to-digital (A/D) converter 304 to be converted to a digital format.

After being converted to a digital format, the signals may be provided via a communication path 306 such as a bus to a storage system 308, or may alternatively be provided directly to a processing circuit 310. Storage system 308 may include any combination of memory or other storage circuits, including Random Access Memory (RAM), Read-Only Memory (ROM), and/or one or more hard disk units. Storage system may store the acquired pressure signals obtained from the patient, and may further store pressure profiles that are indicative of typical pressure measurements obtained at various locations within the heart and associated vascular system. These pressure profiles contain various pressure measurements that are correlated with locations within the heart and associated vascular system. By comparing an acquired pressure measurement with these stored estimated pressure indications included in a pressure profile, an estimate of distal tip location of an IMD may be obtained.

Pressure profiles may be customized for a given individual. For example, using a fluoroscope, pressure measurements may be obtained at precise locations within a patient's body. These measurements and the associated location data may be stored for later use. This data may be employed with a device according to the current invention so that radiopaque dye is no longer needed in subsequent IMD placement procedures.

In another embodiment, a stored pressure profile may be selected for use based on predetermined patient characteristics. For example, a particular pressure profile that is known to correspond to a very large person having heart disease may be selected because it closely matches the patient's characteristics. Using pressure profiles that correspond to patient characteristics allows for a more accurate estimation of device location. If desired, storage system 308 may store many different pressure profiles, or a selected pressure profile may be loaded into the storage system prior to use. In yet another embodiment, the pressure profile can be a profile that is “generic”, but is calibrated for a given user based on one or more initial pressure measurements obtained at the start of a procedure. Other embodiments and uses of the pressure profiles are possible within the scope of the current invention.

As noted above, pressure monitor further includes processing circuit 310, which performs the processing steps to execute the inventive method of the current invention. This includes performing any processing of the newly-acquired pressure measurements, as well as comparing these pressure measurements to the pressure profiles to obtain an estimated location of the IMD distal tip. Processing circuit 310 may be a microprocessor, or any other combination of discrete or integrate components, including a state machine. Processing circuit may execute programmable instructions stored within storage system 308.

Pressure monitor 30 may include an interface circuit 312 that couples to one or more user interface devices 314. Interface circuit may control the flow of data signals from processing circuit 310 and/or storage system 308 to user interface device 314, for example. User interface device(s) 314 may include a display screen and/or any other type of user display such as an LED display. A keyboard or other input device may be provided, along with an audio output or input, and/or any other type of user input/output device known in the art.

In one embodiment, a display screen provides some indication of the approximate location of a distal tip of an IMD. For example, a diagram of a heart and associated cardiovascular system may be displayed together with a depiction of a catheter, lead, sheath, or other device so that the user can determine the approximate location of the IMD distal tip. In another embodiment, the pressure signal may itself be displayed instead of, or in addition to, the physiological depiction in the manner discussed above. Any other type of indication that provides the user with an estimation of IMD location to aid in navigation may be used in addition to, or instead of, the above-described exemplary indications.

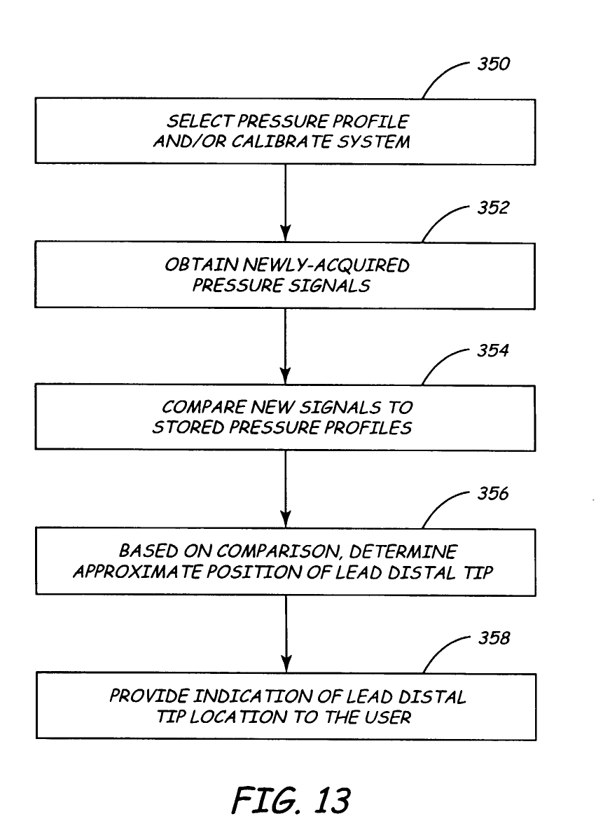

FIG. 13 is a method of placing an implantable medical device within a body according to the current invention. First, a given pressure profile may be selected for a given patient, and/or calibration may be performed to adjust an existing pressure profile to the particular patient (350). This may involve obtaining several initial pressure measurements within the IMD system after the system is introduced into the patient's body. Next, pressure signals may be obtained as the IMD is adapted within the body (352). These acquired signals are compared to the stored signals in the selected pressure profile to obtain estimates of location of the device distal tip within the body (354, 356). This estimation is used to provide the user with an indication of the distal tip location. (358). The indication may include a visual rendition of the patient's anatomy superimposed with a rendition of the IMD. In another embodiment, waveform displays, digital readouts, and or other information may be provided to the user.

The current invention provides a system and method for accurately positioning IMDs within the heart or vascular system without utilizing a fluoro visible media. Those skilled in the art will recognize that many variations of this system and method are possible within the scope of the invention. For example, multiple pressure transducers may be incorporated along a body of an IMD so that location estimates may be obtained for various portions of the IMD. In one embodiment, a transducer may be located at a point other than at a distal tip of a device. As mentioned above, measurements may also be obtained using a pressure measurement system that measures pressure by employing a column of fluid disposed within a lumen of the device. In this instance, the pressure transducer may be located at a proximal end of the device. Therefore, the above embodiments are to be considered exemplary in nature only, with the scope of the invention being limited only by the claims that follow.

What is claimed is:

1. A system for positioning an implantable medical device (IMD) within a living body, comprising:
an elongated body;
a flow-directed member coupled to the elongated body; and
a pressure measuring device coupled to the elongated body to obtain pressure measurements.

2. The system of claim 1, and further comprising a pressure monitor coupled to the pressure measuring device to utilize the pressure measurements to estimate a location of one or more portions of the elongated body relative to the living body.

3. The system of claim 2, wherein the elongated body includes a proximal and distal end, and wherein the pressure measuring device includes means for obtaining pressure measurements at the distal end, whereby the relative location of the distal end of the elongated body may be estimated.

4. The system of claim 2, wherein the flow-directed member is an inflatable device.

5. The system of claim 2, wherein the elongated body includes at least one pull-wire to accomplish deflection of a portion of the elongated body.

6. The system of claim 2, wherein the elongated body includes a lumen to receiving a stiffening member.

7. The system of claim 2, wherein the elongated body includes at least one electrode.

8. The system of claim 2, wherein the proximal end includes marker bands to indicate the location of the distal end within the body.

9. The system of claim 1, wherein the pressure monitor includes a processing circuit.

10. The system of claim 9, wherein the pressure monitor includes a storage system coupled to the processing circuit to store at least one pressure profile, wherein the pressure profile is used to correlate ones of the pressure measurements to locations within the living body.

11. The system of claim 10, wherein the pressure monitor includes a user interface to provide an indication to a user of the estimated location of one or more portions of the elongated body relative to the living body.

12. The system of claim 11, wherein the user interface includes a display screen.

13. The system of claim 2, wherein the elongated body is the body of an IMD selected from the group consisting of a lead, a catheter, and a sheath.

14. An implantable medical lead for implantation in a living body, comprising:
an elongated body;
a flow-directed member coupled to the elongated body; and
a pressure measurement device coupled to the elongated body to obtain pressure measurements at one or more locations within the living body adjacent to the elongated body.

15. The lead of claim 14, and further including a pressure monitor coupled to the pressure measurement device to utilize the pressure measurements to provide an indication of a location within the living body of at least a portion of the implantable medical lead.

16. The lead of claim 15, wherein the flow-directed member is an inflatable member.

17. The lead of claim 16, wherein the elongated body includes a proximal and a distal end, and wherein the lead includes a fixation member coupled to the distal end.

18. The lead of claim 17, wherein the inflation member is positioned to inflate adjacent to at least a portion of the fixation member.

19. The lead of claim 18, wherein the inflation member is shaped to at least partially surround the fixation member when in an inflated state.

20. The lead of claim 19, wherein the fixation member includes a helix.

21. The lead of claim 18, wherein the fixation member includes one or more tines.

22. A sheath for use in implanting an implantable medical device within a living body, comprising:
an elongated body having a proximal end and a distal end, and an inner lumen to receive the implantable medical device;
a flow-directed member coupled to the distal end;
a pressure measuring device coupled to the elongated body to measure pressure at one or more predetermined points adjacent the elongated body when the elongated body is located within the living body; and
a pressure monitor coupled to the pressure measuring device to estimate a position of at least a portion of the elongated body relative to the living body.

23. The sheath of claim 22, and further including an inflatable member coupled to the elongated body adapted to compress the inner lumen and grip the implantable medical device positioned within the lumen.

24. The sheath of claim 23, wherein the elongated body is formed of a material having sufficient stiffness to transfer torque from the proximal end to the distal end.

25. The sheath of claim 22, and further including at least one pull-wire incorporated into the elongated body to deflect the distal end.

26. A method of positioning an implantable medical device (IMD) within a living body, wherein the IMD includes a flow-directed member and a pressure measuring device, comprising:
a.) introducing a portion of the IMD into the living body;
b.) deploying the flow-directed member;
c.) utilizing the pressure measuring device to obtain one or more pressure measurements; and
d.) utilizing the one or more pressure measurements to position the IMD within the living body.

27. The method of claim 26, wherein step b.) comprises inflating an inflatable member.

28. The method of claim 27, wherein step d.) comprises:
d1. ) providing a pressure profile including pressure data correlated to locations within the living body;
d2.) comparing the one or more pressure measurements obtained in step b.) to the pressure data; and
d3.) utilizing the results of the comparison to estimate a location within the living body at which the one or more pressure measurements were obtained.

29. The method of claim 28, wherein step d3) comprises providing an indication of the estimated location for use in positioning the IMD.

30. The method of claim 29, wherein the indication of the estimated location includes a visual representation of at least a portion of the living body and at least a portion of the IMD.

31. The method of claim 29, wherein the indication of the estimated location includes a pressure waveform.

32. The method of claim 26, wherein the IMD is a guide catheter having a lumen, and further including the steps of:
providing a medical electrical lead;
advancing the lead within the lumen of the guide catheter; and
withdrawing the guide catheter from the body, leaving the lead in position.

33. The method of claim 32, wherein the guide catheter includes a distal end, wherein step (d.) includes deflecting the distal end.

34. The method of claim 26, wherein the IMD is a sheath having a lumen, and further 4 including the steps of:
providing a medical electrical lead; and
advancing the lead within the lumen of the sheath.

35. The method of claim 34, wherein the sheath includes an inflation member, and further comprising:
inflating the inflation member to grip the lead positioned within the lumen of the sheath;
deploying the lead within the living body; and
withdrawing the sheath from the living body.

36. The method of claim 27, wherein the IMD is a lead having a fixation member, and further comprising inflating the inflatable member adjacent to the fixation member to substantially prevent the fixation member from contacting tissue within the living body.

37. The method of claim 36, and further comprising
deflating the inflatable member after the lead is substantially located at a predetermined position within the living body; and
affixing the fixation member to tissue within the living body.
